(19) United States
(12) Patent Application Publication
(10) Pub. No.: US 2007/0059751 A1
(43) Pub. Date: Mar. 15, 2007

(54) Nucleic acid isolation method

(75) Inventors: Yoshihiro Yamashita, Hitachinaka (JP); Toshinari Sakurai, Hitachinaka (JP)

Correspondence Address: MCDERMOTT WILL & EMERY LLP, 600 13TH STREET, N.W., WASHINGTON, DC, 20005-3096, US

(73) Assignee: HITACHI HIGH-TECHNOLOGIES CORPORATION

(21) Appl. No.: 11/518,175

(22) Filed: Sep. 11, 2006

(30) Foreign Application Priority Data
Sep. 12, 2005 (JP) 2005-263168

(51) Int. Cl. C12Q 1/68; C12N 1/08

(52) U.S. Cl. 435/6; 435/270

(57) ABSTRACT

In a nucleic acid isolation method for a solid biological sample, since two or more kinds of instruments are used for biological sample disruption and nucleic acid isolation, the operations are complicated, thereby increasing the operating labor, prolonging the operation time, and deteriorating the property of a nucleic acid associated with the prolonged operation time. A sample stuck to the instrument for disruption during the sample disruption operation is not brought to the subsequent nucleic acid isolation operation, thereby causing a problem of reducing the nucleic acid isolation efficiency. In the present nucleic acid isolation method, a step of disrupting a biological sample and a step of isolating a nucleic acid released from the disrupted sample are conducted with one instrument. The nucleic acid isolation efficiency can be improved without losing a sample stuck to an instrument for sample disruption, and the operability can be improved by simplifying the operations.

### BACKGROUND OF THE INVENTION

1. Field of the Invention

The present invention relates to a nucleic acid isolation technology from a biological sample containing a nucleic acid. For example, the present invention relates to a method by which an operation of disrupting a solid biological sample containing a nucleic acid and an operation of isolating the nucleic acid released from the disrupted biological sample are consistently carried out with the use of a single instrument.

2. Background Art

Gene information obtained through the analysis of nucleic acids is utilized in various fields such as medical care, clinical examination, pharmaceutical industry, and food industry. For such nucleic acid analysis, nucleic acid isolation from various biological samples is indispensable as a pretreatment.

As a recent nucleic acid isolation method, the following methods are generally used: the method based on the property of a nucleic acid binding to silica in the presence of a chaotropic agent as reported in [B. Vogelstein and D. Gillespie, Proc. Natl. Acad. Sci. USA, 76 (2), 615-619 (1979)] and the method based on the property of a nucleic acid binding to silica in the presence of an organic solvent as reported in JP Patent Publication (Kokai) No. 2001-95572 A and JP Patent Publication (Kokai) No. 2002-360245 A, instead of a method involving a harmful organic solvent such as phenol or chloroform.

Meanwhile, in a general nucleic acid isolation method from a solid biological sample such as animal and plant tissues or cultured cells, a biological sample is subjected to a chemical dissolution treatment using a chaotropic agent, protein denaturing agent, surface active agent, and the like and is subject to a physical disruption treatment using a homogenizer, a bead mill, a pestle, a syringe provided with an injection needle, a test tube mixer, and the like, so that a nucleic acid contained in the biological sample is released. The nucleic acid is then isolated by utilizing the binding property between silica and a nucleic acid.
Patent Document 1: JP Patent Publication (Kokai) No. 2001-95572 A
Patent Document 2: JP Patent Publication (Kokai) No. 2002-360245 A
Non-patent Document 1: B. Vogelstein and D. Gillespie, Proc. Natl. Acad. Sci. USA, 76 (2), 615-619 (1979)

### SUMMARY OF THE INVENTION

In the above nucleic acid isolation methods for a solid biological sample, since two or more kinds of instruments are used for biological sample disruption and nucleic acid isolation, the operations thereof are complicated, thereby increasing the operating labor, prolonging the operation time, and deteriorating the property of the nucleic acid associated with the prolonged operation time. Furthermore, during the operation of disrupting a biological sample, a sample stuck to the instrument for disruption is not brought to the next process, that is, the nucleic acid isolation operation, thereby causing a problem of deteriorating the efficiency of nucleic acid isolation.

The present invention relates to a nucleic acid isolation method comprising a step of disrupting a biological sample and a nucleic acid isolation step of isolating the nucleic acid that is released from the disrupted sample, and the two steps are carried out with a single instrument.

In accordance with the present invention, the efficiency of nucleic acid isolation can be improved without loss of a sample stuck to an instrument for sample disruption, and the operability can be improved by simplifying the operations.

### BRIEF DESCRIPTION OF THE DRAWINGS

FIG. 1 shows a schematic diagram of an instrument for nucleic acid isolation.

FIG. 2 shows a schematic diagram of an instrument for nucleic acid isolation.

FIG. 3 shows a schematic diagram of opening/closing plates for a solution passing hole (open in the figure).

FIG. 4 shows a schematic diagram of the opening/closing plates for a solution passing hole (closed in the figure).

FIG. 5 shows a cross-sectional view of the instrument for nucleic acid isolation.

FIG. 6 shows an exploded diagram of the instrument for nucleic acid isolation.

### DESCRIPTION OF PREFERRED EMBODIMENTS OF THE INVENTION

The instrument comprises a tip having an end structure or a thin tube structure capable of efficiently disrupting a biological sample, and a solid phase carrier for binding a nucleic acid is fixed inside the tip. Further, solution can be allowed to pass through the inside of the tip and the inside of the solid phase carrier with the use of an instrument for pressure control connected to the tip. Alternatively, the instrument may comprise a syringe connected to the tip having an end structure or a thin tube structure capable of efficiently disrupting a biological sample, and the solid phase carrier for binding a nucleic acid fixed inside the syringe.

The tip end structure capable of efficiently disrupting a biological sample refers to a shape such that the tip end portion can be brought into contact with the lowermost surface of a container into which a sample is added, and a sample can be disrupted by, for example, rotary, circular, or vertical motions of the tip end portion in a state where the tip end portion is pressed against the biological sample in the container. However, it is noted that when a biological sample is disrupted by the above method, it is necessary to adopt an openable and closable a mechanism to a solution passing hole and to carry out a disruption step with the solution passing hole being closed, so as to prevent the biological sample from being stuck in the solution passing hole at the tip end.

The rotary, circular, or vertical motion of the tip end portion may be carried out manually, or it may be driven by a motor.

As a preferred mechanism that opens/closes the solution passing hole at the tip end, at least one opening/closing plate for the solution passing hole is provided at the tip end portion, so that when the tip end is pressed against the bottom surface of a container, the opening/closing plate is folded in the solution passing hole direction due to elastic deformation, whereby the solution passing hole is closed by being covered with the plate. On the other hand, when the tip end portion is separated from the bottom surface of the container, the solution passing hole becomes open again as the opening/closing plate is separated from the solution passing hole. With such opening/closing mechanism, even when a disrupted sample becomes stuck in a minute gap between the closed solution passing hole and the opening/closing plate during sample disruption, the sample that has been stuck can be removed by separating the tip end from the bottom surface of the container and opening the solution passing hole.

Further, the thin tube structure capable of efficiently disrupting a biological sample refers to a thin-tubular solution passing path provided inside the tip, and the structure has a shape such that a sample can be disrupted by allowing the sample to pass through the thin tube as the sample is aspirated into/discharged out of the tube a plurality of times with the use of the instrument for pressure control connected to the tip. Preferably, the internal diameter of the thin tube is approximately between 0.5 and 1.5 mm.

The solid phase carrier fixed inside the tip or inside the syringe is a porous solid phase composed of a substance containing a silicon oxide such as glass fiber filter, glass particles, silica particles, silica wool, disrupted matter thereof, or diatomaceous earth. Preferably, the maximum pore size is between 2 and 20 μm.

Examples of the solid biological sample include animal tissues, plant tissues, bacteria, and cells.

The step of disrupting a biological sample includes either or both of the following steps: a step of disrupting a sample by allowing rotary, circular, or vertical motion of the tip end portion in a state where the tip end portion of the instrument is pressed against the sample to which a lysis agent for chemically or biochemically accelerating the lysis of the biological sample is added; and a step of disrupting a sample by allowing a sample to which the lysis agent is added to pass through the thin-tubular solution passing path inside the tip of the instrument with the use of the instrument for pressure control.

The lysis agent includes a chaotropic agent such as sodium iodide, potassium iodide, sodium thiocyanate, guanidine thiocyanate, or guanidine hydrochloride. To such lysis reagent, a surface active agent or a protein-degrading enzyme may be added. Such chaotropic agent lyses a biological sample due to the protein denaturation effect, inactivates a nuclease derived from the biological sample, and accelerates the binding between silica and the nucleic acid in the subsequent nucleic acid isolation step.

The nucleic acid isolation step of isolating a nucleic acid from a disrupted biological sample comprises a step of allowing a nucleic acid released from a biological sample to bind to a solid phase carrier, a step of washing the nucleic acid that has bound to the solid phase carrier, and a step of eluting the nucleic acid that has bound to the solid phase carrier.

The step of allowing a nucleic acid to bind to a solid phase carrier is carried out by passing a solution, in which a binding agent is added to an lysis reagent containing a nucleic acid released from a biological sample, from one space formed by the solid phase carrier fixed inside the tip or inside the syringe to the other space through the inside of the solid phase carrier, with the use of the instrument for pressure control.

The binding agent refers to an aqueous solution containing an organic solvent.

As the organic solvent, one kind of compound having 2 to 10 carbon atoms selected from aliphatic alcohol, aliphatic ether, aliphatic ester, and aliphatic ketone, or a combination of two or more kinds thereof can be used.

Preferably, ethanol, isopropanol, propanol, or butanol is used as the aliphatic alcohol.

Preferably, ethylene glycol dimethyl ether, ethylene glycol diethyl ether, propylene glycol dimethyl ether, propylene glycol diethyl ether, diethylene glycol dimethyl ether, or diethylene glycol diethyl ether is used as the aliphatic ether.

Preferably, propylene glycol monomethyl ether acetate or ethyl lactate is used as the aliphatic ester.

Preferably, acetone, hydroxyacetone, or methyl ketone is used as the aliphatic ketone.

In the step of washing a nucleic acid that has bound to a solid phase carrier, impurities absorbed to the solid phase carrier are removed by passing a washing reagent from one space formed by the solid phase carrier fixed inside the tip or inside the syringe to the other space through the inside of the solid phase carrier, with the use of the instrument for pressure control. As the washing reagent, a buffer solution of low salt concentration containing an organic solvent such as ethanol is used, for example, so as to prevent the elution of the nucleic acid that has bound to the solid phase carrier and to remove nonspecifically absorbed impurities efficiently. A chaotropic agent or a surface active agent may be added to the buffer solution of low salt concentration containing an organic solvent such as ethanol.

In the step of eluting a nucleic acid that has bound to a solid phase carrier, the nucleic acid that has bound to the solid phase carrier is eluted by passing an elution reagent from one space formed by the solid phase carrier fixed inside the tip or inside the syringe to the other space through the inside of the solid phase carrier, with the use of the instrument for pressure control, and the reagent is then collected as a purified nucleic acid reagent. A pure water, a buffer solution of low salt concentration, or the like that has been subjected to a nuclease removal treatment or a nuclease inactivation treatment is used as the elution reagent, so as to elute the nucleic acid that has bound to the solid phase carrier efficiently and to prevent degradation of the nucleic acid.

### Example 1

RNA isolation from a biological sample was carried out using an instrument for nucleic acid isolation capable of consistently performing the operation of disrupting a biological sample and the operation of isolating the nucleic acid released from the biological sample. The concentration determination and purity calculation of the isolated RNA were conducted with a spectrophotometer. The instrument for nucleic acid isolation, reagents, containers, a biological sample, and a protocol used in the present example will be described in the following. As comparative example 1 against the present example, RNA isolation was carried out by conducting the nucleic acid isolation step alone using the above instrument after conducting the disruption step using general instruments for sample disruption. In the following, the instrument for sample disruption and the protocol used in the method for comparison will also be described.

<Instrument for Nucleic Acid Isolation>

The instrument for nucleic acid isolation comprises a tip in which a solid phase carrier is fixed and an opening/closing plate for a solution passing hole at an end portion of a pipette tip. Hereafter, the instrument for nucleic acid isolation will be described in detail.

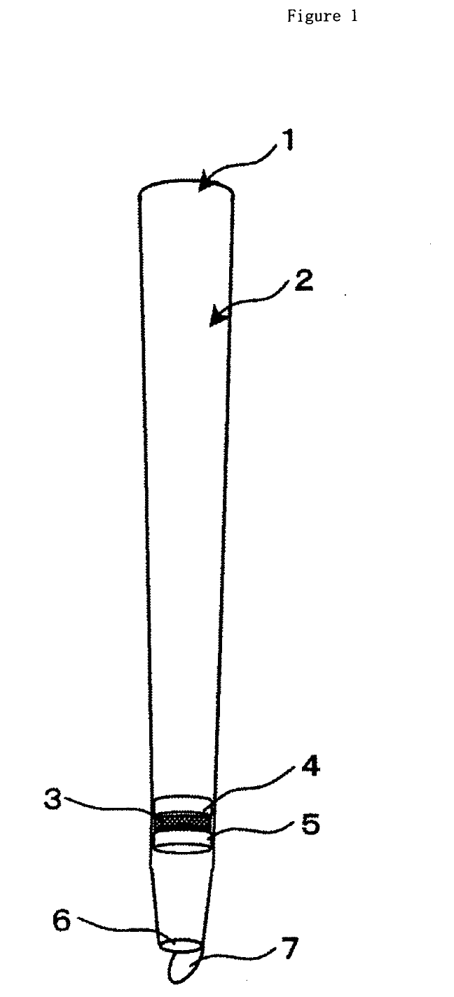

As shown in FIG. 1, the instrument for nucleic acid isolation comprises a nucleic acid-binding solid phase carrier 3 and solid phase carrier holding members 4 and 5 that are fixed inside the end portion of a tip body 2 having a connecting portion 1 connectable to an instrument for pressure control. Further, the instrument comprises a solution passing hole 6 and an opening/closing plate 7 for the solution passing hole at the end portion.

The tip body is made by processing a pipette tip made of polypropylene, and the connecting portion connectable to the instrument for pressure control can be connected to a pipetter or a syringe. The solid phase carrier is a glass fiber filter (GMF 150 manufactured by Whatman Ltd.) that has been cut out with a punch type cutter so that it is shaped to be circular having the same diameter as the internal diameter of the tip. The solid phase carrier holding members are porous members shaped to be circular having the same diameter as the internal diameter of the tip by sintering polypropylene particles. The members fix and hold the solid phase carrier, and they also act as a pre-filter when solution passes through the solid phase carrier. The opening/closing plate for the solution passing hole used for sample disruption is an elliptical plate added to the circumferential portion of the solution passing hole. When the end portion of the opening/closing plate for the solution passing hole is pressed against the bottom surface of a container, the opening/closing plate is caused to bend in the solution passing hole direction due to elastic deformation, and the solution passing hole is closed by being covered with the plate. Thus, the opening/closing plate is firmly attached to the bottom surface of the container. When the end portion is separated from the bottom surface of the container, the opening/closing plate is separated from the solution passing hole, and thus the hole becomes open again.

<Reagents>

Chaotropic solution: 4M guanidine thiocyanate, 8 mM MES-KOH (pH 5.5)

Organic solvent: 50% diethylene glycol dimethyl ether solution

Washing reagent: 80% EtOH solution

Elution reagent: RNase Free sterile pure water

<Containers>

Container for isolation: 2.0 ml microtube (polypropylene)

Container for isolated material: 1.5 ml microtube (polypropylene)

<Biological Sample>

Mouse liver

<Protocol>

1. 5 mg of mouse liver tissue was weighed into the container for isolation.
2. 0.5 ml of chaotropic lysis reagent was added into the container for isolation.
3. The end portion of the instrument for nucleic acid isolation was brought into contact with the bottom surface of the container and moved circularly, the end portion was separated from the bottom surface of the container after a while, and the reagent was aspirated/discharged in such a manner that the reagent would not come into contact with the solid phase carrier.
4. 0.5 ml of organic solvent was added into the container for isolation and mixed.
5. The mixed reagent was aspirated into and discharged out of the instrument for nucleic acid isolation five times in such a manner that the reagent passed through the solid phase carrier, and the reagent was discarded.
6. 1.5 ml of washing reagent was aspirated into and discharged out of the instrument for nucleic acid isolation three times, so as to discard the reagent.
7. 1.5 ml of washing reagent was aspirated into and discharged out of the instrument for nucleic acid isolation three times, so as to discard the reagent.
8. 10 ml of washing reagent was aspirated into and discharged out of a syringe 1 for isolation three times, so as to discard the reagent.
9. 1.5 ml of washing reagent was aspirated into and discharged out of the instrument for nucleic acid isolation three times, so as to discard the reagent. 10. 0.1 ml of elution reagent was aspirated into and discharged out of the instrument for nucleic acid isolation ten times, so as to discharge the reagent into the container for purified nucleic acid solution.
   <Instrument for Sample Disruption in Comparative Example 1>

Pestle

<Protocol in Comparative Example 1>

1. 5 mg of mouse liver tissue was weighed into the container for isolation.
2. 0.5 ml of chaotropic lysis reagent was added into the container for isolation.
3. The pestle was prepared.
4. The pestle was inserted into the container and was circularly moved in a state where an end portion of the pestle was maintained to be in contact with the bottom surface of the container.
5. The sample and the reagent stuck to the pestle were placed back into the container as much as possible, and the pestle was discarded.
6. 0.5 ml of organic solvent was added into the container for isolation and mixed.
7. The mixed reagent was aspirated into and discharged out of the instrument for nucleic acid isolation five times in such a manner that the reagent passed through the solid phase carrier, so as to discard the reagent.
8. 1.5 ml of washing reagent was aspirated into and discharged out of the instrument for nucleic acid isolation three times, so as to discard the reagent.
9. 1.5 ml of washing reagent was aspirated into and discharged out of the instrument for nucleic acid isolation three times, so as to discard the reagent.
10. 10 ml of washing reagent was aspirated into and discharged out of a syringe 1 for isolation three times, so as to discard the reagent.
11. 1.5 ml of washing reagent was aspirated into and discharged out of the instrument for nucleic acid isolation three times, so as to discard the reagent.
12. 0.1 ml of elution reagent was aspirated into and discharged out of the instrument for nucleic acid isolation ten times, so as to discharge the reagent into the container for purified nucleic acid solution.
   <Isolation Results>

The results of RNA isolation in example 1 and comparative example 1 are shown in the following. The RNA yield of example 1 shows a value higher than that of the comparative example. This is due to the fact that the sample stuck to the pestle was lost after the sample disruption step in accordance with the method used in the comparative example, while the sample was not lost after the sample disruption step in accordance with example 1. Meanwhile, the value of RNA purity of the example 1 showed approximately the same as that of comparative example 1. Further, the operation of example 1 was simpler and easier than that of the comparative example, and thus the operability was superior to that of comparative example.

TABLE 1

| | RNA Yield | RNA Purity |
|---|---|---|
| Example 1 | 18 μg | 1.89 |
| Comparative Example 1 | 14 μg | 1.88 |

### Example 2

RNA isolation from a biological sample was carried out using an instrument for nucleic acid isolation capable of consistently performing the operation of disrupting a biological sample and the operation of isolating a nucleic acid released from the biological sample. The concentration determination and purity calculation of the isolated RNA were conducted using a spectrophotometer. The instrument for nucleic acid isolation, reagents, containers, biological sample, and protocol used in the present example will be described. As comparative example 1 against the present example, RNA isolation was carried out by conducting the nucleic acid isolation step alone using the instrument used in example 2, as in the comparative example of example 1.

<Instrument for Nucleic Acid Isolation>

The instrument for nucleic acid isolation differs from the instrument used in example 1 in the shape of the opening/closing plate for the solution passing hole.

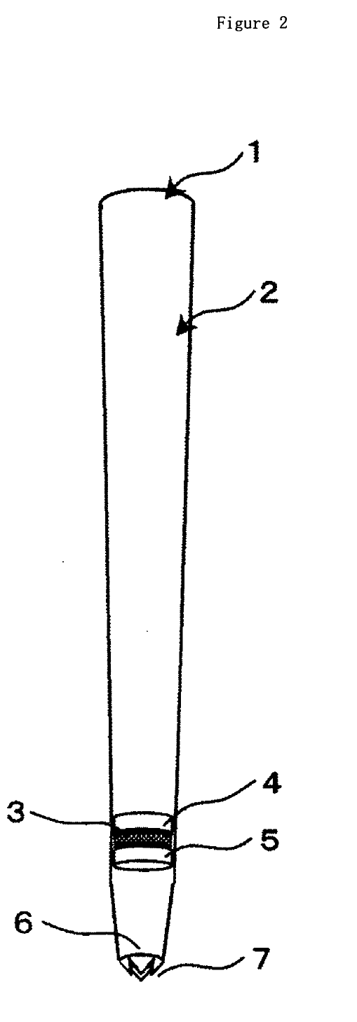

As shown in FIG. 2, the instrument for nucleic acid isolation comprises a nucleic acid-binding solid phase carrier 3 and solid phase carrier holding members 4 and 5 that are fixed inside the end portion of a tip body 2 having a connecting portion 1 connectable to an instrument for pressure control. Further, the instrument comprises a solution passing hole 6 and opening/closing plates 7 for the solution passing hole at the end portion. The connecting portion, tip body, solid phase carrier, solid phase carrier holding members, and solution passing hole are the same as those used in example 1.

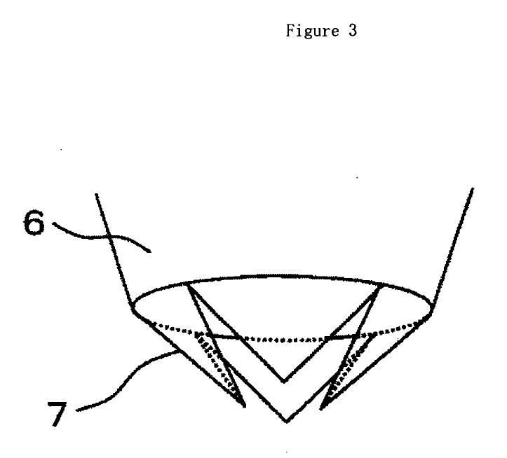

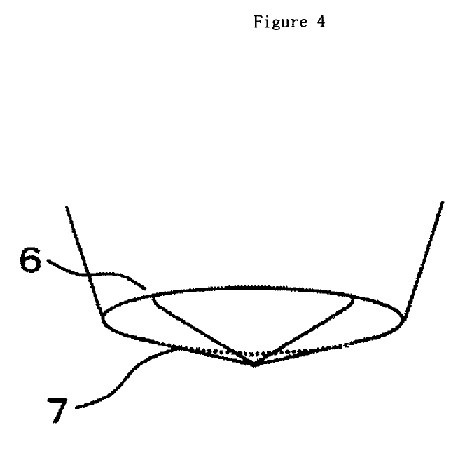

As shown in FIG. 3, the opening/closing plates 7 for solution passing hole are four triangular plates added to the peripheral portion of the solution passing hole. As shown in FIG. 4, when the tip end portion is pressed against the bottom surface of a container, the opening/closing plates for the solution passing hole are bent in the center direction of the solution passing hole due to elastic deformation. The individual edges of the plates are brought into contact with one another, and the solution passing hole is covered with the plates, whereby the solution passing hole is closed and the opening/closing plates are closely attached to the bottom surface of the container. When the tip end portion is separated from the bottom surface of the container, the opening/closing plates are separated from the solution passing hole, whereby the hole becomes open again.

<Biological Sample>

The same as that used in example 1.

<Reagents>

The same as those used in example 1.

<Containers>

The same as those used in example 1

<Protocol>

The same as that of example 1, except for the method for operating the step shown in the following.
3. The end portion of the instrument for nucleic acid isolation was moved vertically and circularly in a state where the end portion was maintained to be in contact with the bottom surface of the container, the end portion was separated from the bottom surface of the container after a while, and the reagent was aspirated/discharged in such a manner that the reagent would not come into contact with the solid phase carrier.
   <Isolation Results>

The results of RNA isolation in example 2 and the comparative example are shown in the following. The RNA yield of example 2 showed a value higher than that of the comparative example and example 1. It can be thought that this is due to the fact that the tissue disruption efficiency was improved due to characteristics of the opening/closing mechanism of the opening/closing plates for the solution passing hole, in addition to the fact that the sample was not lost after the sample disruption step in accordance with example 2. By using the instrument for nucleic acid isolation used in the present method, when the opening/closing plates for the solution passing hole are pressed against the bottom surface of the container by moving the instrument vertically, the sample is efficiently scraped together and crushed on the bottom surface of the container. Further, when the individual edges of the opening/closing plates come into contact with one another, the sample is efficiently disrupted by being sandwiched and crushed. Meanwhile, the value of RNA purity of the example 2 showed approximately the same as that of the comparative example. As in the case of example 1, the operation of example 2 was simpler and easier than that of the comparative example, and thus the operability was superior to that of comparative example.

TABLE 2

| | RNA Yield | RNA Purity |
|---|---|---|
| Example 2 | 20 μg | 1.90 |
| Comparative Example 1 | 14 μg | 1.88 |

RNA Yield RNA Purity

### Example 3

RNA isolation from a sample was carried out using an instrument for nucleic acid isolation capable of consistently performing the operation of disrupting a biological sample and the operation of isolating a nucleic acid released from the biological sample. The concentration determination and purity calculation of the isolated RNA were conducted using a spectrophotometer. The instrument for nucleic acid isolation, reagents, containers, biological sample, and protocol used in the present example will be described. As comparative example 2 against the present example, RNA isolation was carried out by conducting the nucleic acid isolation step alone using the above instrument after conducting the disruption operation using general sample instruments. The sample instrument and the protocol used in the method for comparison will also be described in the following.

<Instrument for Nucleic Acid Isolation>

The instrument for nucleic acid isolation includes a syringe in which a solid phase carrier is fixed and to which a tip including an opening/closing plate for the solution passing hole and a thin tube solution passing path is connected. The instrument will be described in detail in the following.

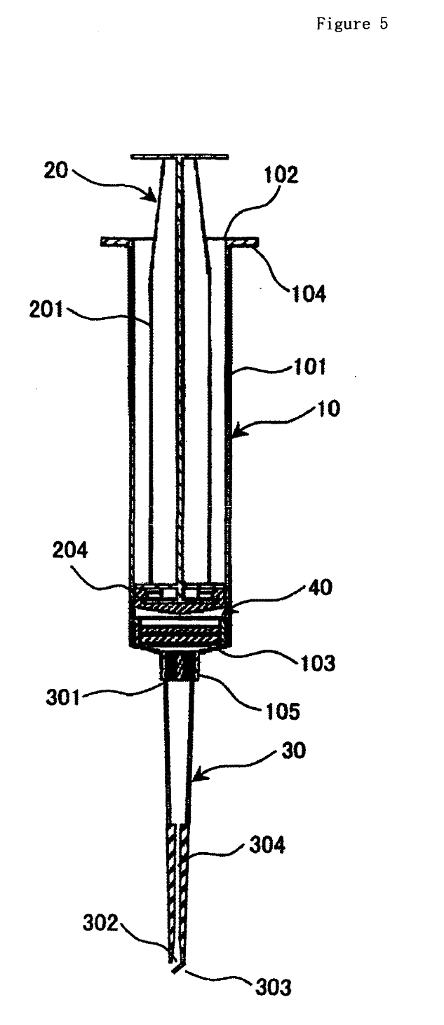

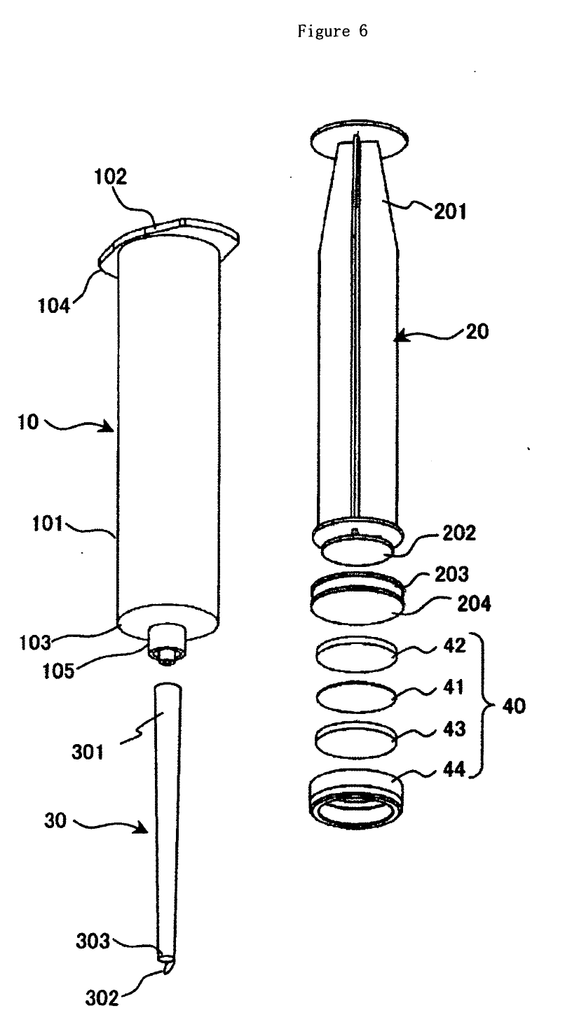

As shown in FIGS. 5 and 6, the instrument for nucleic acid isolation comprises a syringe body 10, a plunger 20, a tip body 30, and a solid phase carrier unit 40.

The syringe body 10 comprises a cylindrical portion 101, an opening 102 at the upper end, a bottom portion 103 at the lower end, a gripping portion 104 having a shape of a sword guard provided at the periphery of the opening 102, and a connecting portion 105 for connecting the tip body provided at the bottom portion 103.

The plunger 20 comprises a plunger body 201 and a seal piece 203. The seal piece 203 is formed as a member separately from the plunger body 201 and attached to an attaching portion 202 at the lower end of the plunger body 201. The seal piece 203 has a conical projection 204 at the lower end thereof The tip body 30 comprises a connecting portion 301 at the upper end, a solution passing hole 302, an opening/closing plate 303 for the solution passing hole, and a thin tube solution passing path 304. The opening/closing mechanism of the opening/closing plate for the solution passing hole is the same as that of the instrument used in example 1. The connecting portion 301 of the nozzle and the connecting portion 105 of the syringe body are connected via a screw.

As shown in FIG. 6, the solid phase carrier unit 40 comprises a disk-shaped solid phase carrier 41, two disk-shaped solid phase carrier holding members 42 and 43 disposed on the upper side and the lower side of the solid phase carrier 41, and a cylindrical holder 44. The solid phase carrier is a glass fiber filter (GMF 150 manufactured by Whatman Ltd.) that has been cut out with a punch type cutter. The solid phase carrier holding members are porous members shaped by sintering polypropylene particles. The members hold the solid phase carrier and also act as a pre-filter when a solution passes through the solid phase carrier.

<Biological Sample>

Mouse kidney

<Reagents>

Chaotropic solution: RLT buffer (manufactured by QIAGEN Inc.)

Organic solvent: 70% ethanol solution

Washing reagent: 80% EtOH solution

Elution reagent: RNase Free sterile pure water

<Containers>

The same as those used in example 1

<Protocol >

1. 5 mg of mouse kidney tissue was weighed into the container for isolation.
2. 0.5 ml of chaotropic lysis reagent was added into the container for isolation.
3. The end portion of the instrument for nucleic acid isolation was brought into contact with the bottom surface of the container and moved circularly, the end portion was separated from the bottom surface of the container after a while, and the reagent was aspirated/discharged in such a manner that the reagent would not come into contact with the thin tube solution passing path.
4. The end portion of the instrument for nucleic acid isolation was separated from the bottom surface of the container, and the reagent was aspirated into and discharged out of the thin tube solution passing hole in such a manner that the reagent would not come into contact with the solid phase carrier.
5. 0.5 ml of organic solvent was added into the container for isolation and mixed.
6. The mixed reagent was aspirated into and discharged out of the instrument for nucleic acid isolation five times in such a manner that the reagent passed through the solid phase carrier, so as to discard the reagent.
7. 1.5 ml of washing reagent was aspirated into and discharged out of the instrument for nucleic acid isolation three times, so as to discard the reagent.
8. 1.5 ml of washing reagent was aspirated into and discharged out of the instrument for nucleic acid isolation three times, so as to discard the reagent.
9. 10 ml of washing reagent was aspirated into and discharged out of a syringe 1 for isolation three times, so as to discard the reagent.
10. 1.5 ml of washing reagent was aspirated into and discharged out of the instrument for nucleic acid isolation three times, so as to discard the reagent.
11. 0.1 ml of elution reagent was aspirated into and discharged out of the instrument for nucleic acid isolation ten times, so as to discharge the reagent into the container for purified nucleic acid solution.
   <Instrument for Tissue Disruption in Comparative Example 2>

Pestle

Syringe provided with an injection needle (20G)

<Protocol in Comparative Example 2>

1. 5 mg of mouse liver tissue was weighed into the container for isolation.
2. 0.5 ml of chaotropic lysis reagent was added into the container for isolation.
3. The pestle was prepared.
4. The pestle was inserted into the container and was circularly moved as an end portion of the pestle was maintained to be in contact with the bottom surface of the container.
5. The sample and the reagent attached to the pestle were placed back into the container as much as possible, and the pestle was discarded.
6. The syringe provided with an injection needle was prepared.
7. The reagent was aspirated into and discharged out of the syringe provided with an injection needle five times.
8. The sample remaining in the injection needle and the syringe were placed back into the container as much as possible, and the injection needle and the syringe were discarded.
9. 0.5 ml of organic solvent was added into the container for isolation and mixed.
10. The mixed reagent was aspirated into and discharged out of the instrument for nucleic acid isolation five times in such a manner that the reagent passed through the solid phase carrier, so as to discard the reagent.
11 1.5 ml of washing reagent was aspirated into and discharged out of the instrument for nucleic acid isolation three times, so as to discard the reagent.
12. 1.5 ml of washing reagent was aspirated into and discharged out of the instrument for nucleic acid isolation three times, so as to discard the reagent.
13. 10 ml of washing reagent was aspirated into and discharged out of a syringe 1 for isolation three times, so as to discard the reagent.
14. 1.5 ml of washing reagent was aspirated into and discharged out of the instrument for nucleic acid isolation three times, so as to discard the reagent.
15. 0.1 ml of elution reagent was aspirated into and discharged out of the instrument for nucleic acid isolation ten times, so as to discharge the reagent into the container for purified nucleic acid solution.
   <Isolation Results>

The results of RNA isolation in example 3 and comparative example 2 are shown in the following. The RNA yield of example 3 showed a value higher than that of the comparative example. It can be thought that this is due to the fact that the sample stuck to the pestle and the syringe provided with an injection needle was lost after the sample disruption step in accordance with the method used in the comparative example, while the sample was not lost after the sample disruption step in accordance with the method in example 3. Meanwhile, the value of RNA purity of the example 3 showed approximately the same as that of comparative example 2. Further, the operation of example 3 was simpler and easier than that of the comparative example that involves two different kinds of instruments in the sample disruption step, and thus the operability was superior to that of the comparative example, whereby the required operation time was shortened.

TABLE 3

| | RNA Yield | RNA Purity |
|---|---|---|
| Example 3 | 17 μg | 1.88 |
| Comparative Example 2 | 12 μg | 1.87 |

What is claimed is:

1. A nucleic acid isolation method for isolating a nucleic acid from a biological sample containing the nucleic acid, comprising:
disrupting a biological sample with the use of a sample disruption mechanism with which an instrument for nucleic acid isolation is provided; and
allowing the nucleic acid released from the disrupted sample to be absorbed to a nucleic acid-binding solid phase with which the instrument for nucleic acid isolation is provided.

2. The nucleic acid isolation method according to claim 1, wherein the instrument for nucleic acid isolation comprises a tube that can be connected to an instrument for pressure control, the inside of which the nucleic acid-binding solid phase is fixed.

3. The nucleic acid isolation method according to claim 1, wherein the instrument for nucleic acid isolation comprises the instrument for pressure control, the inside of which the nucleic acid-binding solid phase is fixed.

4. The nucleic acid isolation method according to claim 1, wherein the sample disruption mechanism with which the instrument for nucleic acid isolation is provided is a hollow thin tube that can be connected to an instrument for pressure control, and a biological sample is disrupted by allowing the biological sample to pass through the tube due to pressure controlled by the instrument for pressure control connected to the tube.

5. The nucleic acid isolation method according to claim 1, wherein the sample disruption mechanism with which the instrument for nucleic acid isolation is provided is a member provided at an end portion of a hollow tube that can be connected to an instrument for pressure control, and when the tube end is pressed against a biological sample in a container, a solution passing hole of the tube is covered and when the tube end is brought into contact with the bottom surface of the container, the biological sample is crushed and disrupted.

6. The nucleic acid isolation method according to claim 1, wherein the sample disruption mechanism with which the instrument for nucleic acid isolation is provided is an opening/closing plate for a solution passing hole provided at an end portion of a hollow tube that can be connected to an instrument for pressure control.
